(19) United States
(12) Patent Application Publication
(10) Pub. No.: US 2003/0229025 A1
(43) Pub. Date: Dec. 11, 2003

(54) Intranasal administration of MC4-R agonists

(75) Inventors: Linda Li Xiao, Alameda, CA (US); Baoji Xu, Rockville, MD (US); Jian Luo, Brisbane, CA (US); Kirk Johnson, Moraga, CA (US); William H. Frey II, White Bear Lake, MN (US); Effie Tozzo, Newton, MA (US); David Duhl, Oakland, CA (US)

Correspondence Address: Steven W. Collier, Chiron Corporation, P.O. Box 8097, Emeryville, CA, 94662, US

(73) Assignee: Chiron Corporation

(21) Appl. No.: 10/374,507

(22) Filed: Feb. 25, 2003

Related U.S. Application Data
(60) Provisional application No. 60/358,700, filed on Feb. 25, 2002.
(60) Provisional application No. 60/372,921, filed on Apr. 16, 2002.

(51) Int. Cl. A61L 9/04; A61K 38/04; A61K 31/495; A61K 31/496; A61K 31/155

(52) U.S. Cl. 514/18; 514/19; 514/634; 424/45; 514/253.01; 514/252.12; 514/254.06; 514/254.09; 514/254.08

(57) ABSTRACT

A method for delivering a melanocortin-4 receptor agonist to a mammalian subject, includes administering the melanocortin-4 receptor agonist to a tissue inside the nasal cavity or sinuses of the mammalian subject. In some instances, the melanocortin-4 receptor agonist includes a guanidine functional group.

### CROSS-REFERENCES TO RELATED APPLICATIONS

This application claims priority to U.S. Provisional Application No. 60/358,700 filed on Feb. 25, 2002, and U.S. Provisional Application No. 60/372,921 filed on Apr. 16, 2002, the entire disclosures of which are hereby incorporated by reference in their entirety and for all purposes.

### FIELD OF THE INVENTION

This invention relates to a method of intranasal delivery of melanocortin-4 receptor (MC4-R) agonists and compositions for use in intranasal delivery of MC4-R agonists. The invention also relates to methods of treating MC4-R-mediated disorders, such as obesity, type II diabetes, or eating disorders, such as bulimia, by activating the melanocortin-4 receptor with compounds and compositions provided herein.

### BACKGROUND OF THE INVENTION

Melanocortins are peptide products resulting from post-translational processing of pro-opiomelanocortin and are known to have a broad array of physiological activities. The natural melanocortins include the different types of melanocyte stimulating hormone (α-MSH, β-MSH, γ-MSH) and ACTH. Of these, α-MSH and ACTH are considered to be the main endogenous melanocortins.

The melanocortins mediate their effects through melanocortin receptors (MC-R), a subfamily of G-protein coupled receptors. There are at least five different receptor subtypes (MC1-R to MC5-R). MC1-R mediates pigmentation of the hair and skin. MC2-R mediates the effects of ACTH on steroidogenesis in the adrenal gland. MC3-R and MC4-R are predominantly expressed in the brain. MC5-R is considered to have a role in the exocrine gland system.

The melanocortin-4 receptor (MC4-R) is a seven-transmembrane receptor. MC4-R may participate in modulating the flow of visual and sensory information, coordinate aspects of somatomotor control, and/or participate in the modulation of autonomic outflow to the heart. Science 257:1248-125 (1992). Significantly, inactivation of this receptor by gene targeting has resulted in mice that develop a maturity onset obesity syndrome associated with hyperphagia, hyperinsulinemia, and hyperglycemia. Cell Jan 10; 88(1): 131-41 (1997). MC4-R has also been implicated in other disease states including erectile disorders, cardiovascular disorders, neuronal injuries or disorders, inflammation, fever, cognitive disorders, and sexual behavior disorders. Hadley M.E. and Haskell-Luevano C., The Proopiomelanocortin System, Ann. N. Y. Acad. Sci., 885:1 (1999).

Furthermore, observations in connection with endogenous MC4-R antagonists indicate that MC4-R is implicated in endogenous energy regulation. For example, an agouti protein is normally expressed in the skin and is an antagonist of the cutaneous MC receptor involved in pigmentation, MC1-R. M. M. Ollmann et al., Science, 278:135-138 (1997). However, overexpression of agouti protein in mice leads to a yellow coat color due to antagonism of MC1-R and increased food intake and body weight due to antagonism of MC4-R. L. L. Kiefer et al., Biochemistry, 36: 2084-2090 (1997); D. S. Lu et al., Nature, 371:799-802 (1994). Agouti related protein (AGRP), an agouti protein homologue, antagonizes MC4-R but not MC1-R. T. M. Fong et al., Biochem. Biophys. Res. Commun. 237:629-631 (1997). Administration of AGRP in mice increases food intake and causes obesity but does not alter pigmentation. M. Rossi et al., Endocrinology, 139:4428-4431 (1998). Together, this research indicates that MC4-R participates in energy regulation, and therefore, identifies this receptor as a target for a rational drug design for the treatment of obesity.

In connection with MC4-R and its uncovered role in the etiology of obesity and food intake, various compounds or compositions that act as agonists or antagonists of MC4-R have been reported. As examples, U.S. Pat. No. 6,060,589 describes polypeptides that are capable of modulating signaling activity of melanocortin receptors. Also, U.S. Pat. Nos. 6,054,556 and 5,731,408 describe families of agonists and antagonists for MC4-R receptors that are lactam heptapeptides having a cyclic structure.

Published PCT applications WO2001/55109, WO2001/55107 and WO2001/55016 disclose aromatic amines and/or amides for the treatment of obesity, anorexia, inflammation, mental disorders and other diseases associated with the melanocortin receptors or related systems. The disclosed amines and amides have been shown to bind to melanocortin receptors (e.g., MC-1, MC-3, MC-4 and/or MC-5) and function as either agonists or antagonists of a specific MC-receptor or of multiple MC-receptors.

U.S. Pat. Nos. 6,180,603 and 6,313,093 disclose the delivery of therapeutic substances to the brain for the treatment of insulin related disorders, as well as neurologic or psychiatric conditions, by intranasal administration of the neurologic agent via the olfactory system of the brain. The neurologic agents that are disclosed are useful in the treatment of brain disorders such as Alzheimer's disease, Parkinson's disease, affective disorders (e.g., depression and mania and nerve damage).

Fehm et al. describe the role of melanocortins in the long-term control of fat stores in humans. Fehm et al. The Journal of Clinical Endocrinology & Metabolism 86:1144-1148(2001). In this study, melanocyte stimulating hormone/adrenocorticotropin₄₋₁₀(MSH/ACTH₄₋₁₀) and desacetyl-αMSH were intranasally administered to various subjects. Fehm et al. discloses that intranasal administration of MSH/ACTH₄₋₁₀ reduced body fat, on the average. Additionally, plasma leptin levels and insulin levels decreased after intranasal administration of MSH/ACTH₄₋₁₀. In contrast, changes after intranasal administration of desacetyl-αMSH remained nonsignificant. According to the authors, the finding of reduced body adiposity after MSH/ACTH₄₋₁₀ confirmed, and extended to the human, the findings of animal models indicating an essential role of the hypothalamic melanocortin system in body weight control.

In another study Smolnik et al. disclose that neuropeptides related to adrenocorticotropin (ACTH) are potent regulators of neurobehavioral functions. Smolnik et al. Neuroendocrinology 70:63-72 (1999). In humans, ACTH and its behaviorally active fragment ACTH₄₋₁₀, have been consistently found to diminish event-related brain potential (ERP) signs of focusing attention. As disclosed by Smolnik et al. acute intranasal administration of ACTH₄₋₁₀ (1 mg) reduced the processing negativity (PN) of the ERP over frontal and central cortical areas indicating diminished focusing of attention. Acute intranasal administration of desacetyl-αMSH at equimolar doses, however, (1.68 mg) is disclosed as being ineffective. The authors report that the effects of intranasal administration are likely to reflect a direct action of the peptide on respective brain functions. Moreover, Smolnik et al. concluded that since the effects were specific to ACTH₄₋₁₀ and were not obtained after equimolar doses of desacetyl-αMSH, a mediation via the known melanocortin receptors was excluded.

### SUMMARY OF THE INVENTION

There is a need for potent and specific agonists of MC4-R that are low molecular weight small molecules and improved methods for admininstering such compounds. Methods of treating a melanocortin-4 receptor mediated disease, such as obesity, type II diabetes and eating disorders such as bulimia, with such drugs, are also particularly desirable. Intranasal delivery is an efficacious method for the administration of MC4-R agonists and for treating MC4-R mediated diseases.

The invention, therefore, relates to a method of treating an MC4-R mediated disease, comprising intranasally administering to a subject in need thereof, a therapeutically effective amount of an MC4-R agonist. In some embodiments, the agonist is a compound with a molecular weight of less than 900 g/mol. In other embodiments, the MC4-R agonist is a compound with a molecular weight of less than 700 g/mol. In yet other embodiments, the MC4-R agonist is a compound with a molecular weight ranging from 450 g/mol to 700 g/mol. In yet other embodiments, the MC4-R agonist is a compound with a molecular weight ranging from 500 g/mol to 700 g/mol. In yet further embodiments, the MC4-R agonist is a compound with a molecular weight of about 600 g/mol. In still other embodiments, the MC4-R agonist includes 3 or less amino acid residues.

The invention further relates to a method of treating an MC4-R mediated disease, comprising intranasally administering to a subject in need thereof, a composition comprising an MC4-R agonist and a pharmaceutically acceptable carrier. In some embodiments, the agonist is a compound with a molecular weight of less than 900 g/mol whereas in other embodiments, the MC4-R agonist is a compound with a molecular weight of less than 700 g/mol. In yet other embodiments, the MC4-R agonist is a compound with a molecular weight ranging from 450 g/mol to 700 g/mol. In yet other embodiments, the MC4-R agonist is a compound with a molecular weight ranging from 500 g/mol to 700 g/mol. In yet further embodiments, the MC4-R agonist is a compound with a molecular weight of about 600 g/mol. In further embodiments, the MC4-R agonist includes 3 or less amino acid residues. In some such embodiments, the method includes intranasally administering an MC4-R agonist that includes a guanidino group.

The invention also relates to treating an MC4-R mediated disease such as obesity, an eating disorder, or type II diabetes.

An effective method is needed for the delivery of compounds which are useful in the treatment of MC4-R-mediated disorders. Testing MC4-R agonists is an important aspect of developing treatments for MC4-R-mediated disorders. Since existing methods of testing possible agonists for the treatment of MC4-R-mediated disorders are of limited benefit, a goal of the present invention is to develop a procedure for the effective delivery of MC4-R agonists to treat an MC4-R-mediated disorder. Another objective is to develop a composition that can effect efficient absorption of the MC4-R agonists.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A and 1B are graphs showing the efficacy of compound 1 when administered intranasally (FIG. 1A) and orally (FIG. 1B).

FIGS. 2A and 2B are graphs showing the efficacy of compound 9 when administered intranasally (FIG. 2A) and orally (FIG. 2B).

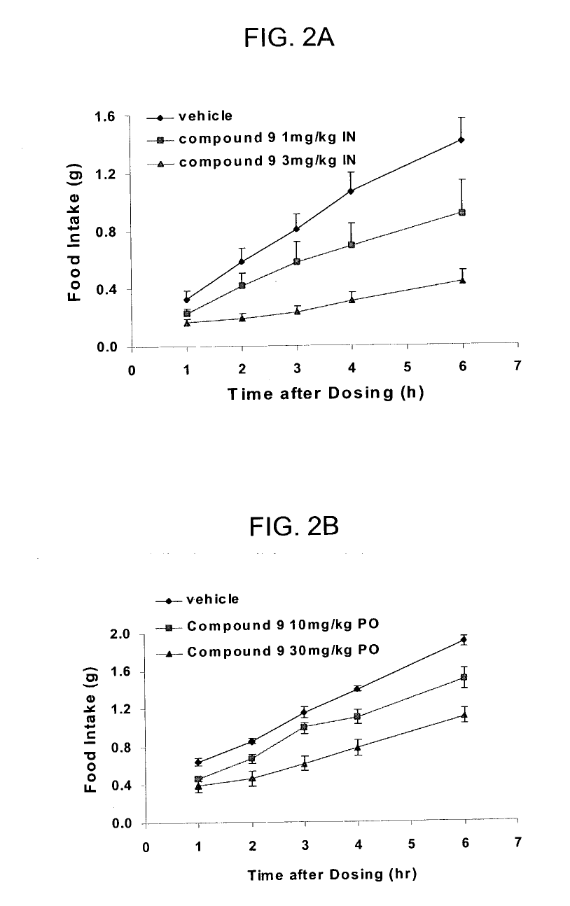

FIGS. 3A and 3B are graphs showing the efficacy of compound 13 when administered intranasally (FIG. 3A) and orally (FIG. 3B).

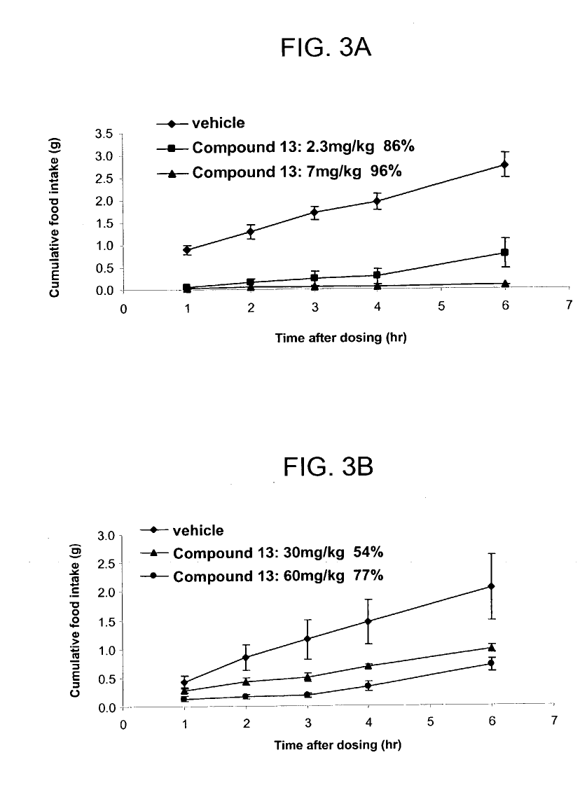

### DETAILED DESCRIPTION OF THE PREFERRED EMBODIMENT

The instant invention provides methods and compositions for the treatment of MC4-R-mediated disorders that comprise the delivery of an MC4-R agonist to a patient in need of such treatment.

I. Intranasal Delivery of MC4-R Agonists

The instant invention provides for methods and compositions to treat MC4-R mediated disorders comprising intranasal delivery of low molecular weight, small molecule agonists of MC4-R. Thus, there has been provided, in accordance with one aspect of the invention, the intranasal delivery of the guanidine derivatives disclosed in U.S. Provisional Application Nos. 60/230,565, filed Aug. 31, 2000; 60/245,579, filed Nov. 6, 2000; 60/282,847, filed Apr. 9, 2001; 60/353,183, filed Feb. 4, 2002; and 60/353,188, filed Feb. 4, 2002. Also contemplated is the intranasal delivery of the guanidine compounds disclosed in U.S. Application Ser. Nos. 09/945,384, filed Aug. 31, 2001,; 10/118,730, filed Apr. 8, 2002; 10/351,574, filed Jan. 27, 2003; and 10/351,597, filed on Jan. 27, 2003. All of the aforementioned applications are hereby incorporated by reference for all purposes, as if fully set forth herein.

The present invention further provides compositions and methods for the intranasal delivery of other low molecular weight, small molecule MC4-R agonist compounds. Particular embodiments include the MC4-R agonists disclosed in WO01/70708 and WO00/74679; the MC4-R agonists disclosed in WO01/70337 and WO99/64002; the MC4-R agonists disclosed in WO01/55109; the MC4-R agonists disclosed in WO01/55107 and WO01/55106; and the MC4-R agonists disclosed in WO01/10842. All of the aforementioned published PCT applications are hereby incorporated by reference for all purposes, as if fully set forth herein.

The compositions and methods of the present invention also include tautomers, prodrugs, pharmaceutically acceptable salts, stereoisomers, hydrates, hydrides, or solvates of any of the MC4-R agonists disclosed in the above-mentioned U.S. Patent Application and Published PCT Applications, alone or in combination.

Stereoisomers include enantiomers, diastereomers, atropisomers and geometric isomers. In some cases, one stereoisomer may be more active and/or may exhibit beneficial effects in comparison to other stereoisomer(s) or when separated from the other stereoisomer(s). However, it is well within the skill of the ordinary artisan to separate, and/or to selectively prepare said stereoisomers. Accordingly, “stereoisomers” of the instant invention necessarily includes mixtures of stereoisomers, individual stereoisomers, or optically active forms.

Prodrugs include those derivatives of said compounds which undergo in vivo metabolic biotransformation, by enzymatic or nonenzymatic processes, such as hydrolysis, to form a compound of the invention. Prodrugs can be employed to improve pharmaceutical or biological properties, as for example solubility, melting point, stability and related physicochemical properties, absorption, pharmacodynamics and other delivery-related properties.

In some embodiments, the MC4-R agonist of a composition for intranasal administration is a compound that has a molecular weight that is less than 900 g/mol, and in other embodiments the MC4-R agonist is a compound with a molecular weight of less than 700 g/mol. In yet other embodiments, the MC4-R agonist is a compound with a molecular weight ranging from 450 g/mol to 700 g/mol. In yet other embodiments, the MC4-R agonist is a compound with a molecular weight ranging from 500 g/mol to 700 g/mol. In yet further embodiments, the MC4-R agonist is a compound with a molecular weight of about 600 g/mol. In some embodiments, the MC4-R agonists are formed from three or less amino acids such that the agonists include three or less amino acid residues.

II. Methods of Administration

The method of the invention administers one or more MC4-R agonists to tissue innervated by the trigeminal and olfactory nerves inside the nasal cavity and/or sinuses.

The trigeminal and olfactory nerve systems can provide a direct connection between the outside environment and the brain, thus providing advantageous delivery of an MC4-R agonist to the central nervous system (CNS), brain, and/or spinal cord.

The Olfactory Nerve

In one aspect, the method of the invention includes administration of an MC4-R agonist to tissue innervated by the olfactory nerve and inside the nasal cavity. Preferably, the agonist is delivered to the olfactory area in the upper third of the nasal cavity and particularly to the olfactory epithelium.

Fibers of the olfactory nerve are unmyelinated axons of olfactory receptor cells that are located in the superior one-third of the nasal mucosa. The olfactory receptor cells are bipolar neurons with swellings covered by hair-like cilia which project into the nasal cavity. At the other end, axons from these cells collect into aggregates and enter the cranial cavity at the roof of the nose. Surrounded by a thin tube of pia, the olfactory nerves cross the subarachnoid space containing cerebrospinal fluid (CSF) and enter the inferior aspects of the olfactory bulbs. Once the agonist is dispensed into the nasal cavity, it can undergo transport through the nasal mucosa and into the olfactory bulb and interconnected areas of the brain (e.g., hippocampal formation, amygdaloid nuclei, nucleus basalis of Meynert, locus ceruleus, the brain stem and the like).

The Trigeminal Nerve

In another aspect, the method of the invention includes administration of an MC4-R agonist to tissue innervated by the trigeminal nerve and inside the nasal cavity. Within the nasal cavity, the trigeminal nerve innervates mainly the inferior two-thirds of the nasal mucosa.

The trigeminal nerve has three major branches: the ophthalmic nerve, the maxillary nerve, and the mandibular nerve. The method of the invention can administer an MC4-R agonist to tissue within the nasal cavity innervated by one or more of these branches.

The Ophthalmic Nerve and its Branches

In yet another aspect, the method of the invention includes administration of an MC4-R agonist to tissue within the nasal cavity and/or sinuses innervated by the ophthalmic nerve branch of the trigeminal nerve. The ophthalmic nerve has three branches known as the nasociliary nerve, the frontal nerve, and the lacrimal nerve. The anterior ethmoidal nerve, a branch of the nasociliary nerve, innervates, among other tissues, the ethmoidal sinus and regions of the interior two-thirds of the nasal mucosa, including the anterior portion of the nasal septum and the lateral wall of the nasal cavity. Preferably, the method of the invention can administer the agonist to tissue innervated by the anterior ethmoidal nerve.

The Maxillary Nerve and its Branches

In still another aspect, the method of the invention can administer an MC4-R agonist to tissue within the nasal cavity and/or sinuses innervated by the maxillary nerve branch of the trigeminal nerve. The maxillary nerve has several branches that innervate the nasal cavity and sinuses, including the nasopalatine nerve, the greater palatine nerve, the posterior superior alveolar nerves, the middle superior alveolar nerve and the interior superior alveolar nerve. The maxillary sinus is innervated by the posterior, middle and anterior superior alveolar nerves. The mucous membrane of the nasal septum is supplied chiefly by the nasopalatine nerve and the lateral wall of the nasal cavity is supplied by the greater palatine nerve. Preferably, the method of the invention can administer an MC4-R agonist to tissue innervated by the nasopalatine nerve and/or greater palatine nerve.

Neuronal Transport

One embodiment of the present method includes administration of an MC4-R agonist to the subject in a manner such that the agonist is transported to the CNS, brain, and/or spinal cord along a neural pathway. A neural pathway includes transport within or along a neuron, through or by way of lymphatics running with a neuron, through or by way of a perivascular space of a blood vessel running with a neuron or neural pathway, through or by way of an adventitia of a blood vessel running with a neuron or neural pathway, or through a hemangiolymphatic system. The invention prefers transport of an MC4-R agonist by way of a neural pathway, rather than through the circulatory system, so that MC4-R agonists that are unable to, or only poorly, cross the blood-brain barrier from the bloodstream into the brain can be delivered to the CNS, brain, and/or spinal cord. The MC4-R agonist, once past the blood-brain barrier and in the CNS, can then be delivered to various areas of the brain or spinal cord through lymphatic channels, through a perivascular space, or transported through or along neurons.

Use of a neural pathway to transport an MC4-R agonist to the brain, spinal cord or other components of the central nervous system obviates the obstacle presented by the blood-brain barrier so that agonists that cannot normally cross that barrier, can be delivered directly to the brain, cerebellum, brain stem or spinal cord. Although the MC4-R agonist that is administered may be absorbed into the bloodstream as well as the neural pathway, the agonist preferably provides minimal effects systemically. In addition, the invention can provide for delivery of a more concentrated level of the MC4-R agonist to neural cells since the agonist does not become diluted in fluids present in the bloodstream. As such, the invention provides an improved method for delivering an MC4-R agonist to the CNS, brain and/or spinal cord. In addition, delivery of a therapeutic, MC4-R agonist to the CNS by a neural pathway can reduce systemic delivery and unwanted systemic side effects.

The Olfactory Neural Pathway

One embodiment of the present method includes delivery of the MC4-R agonist to the subject in a manner such that the agonist is transported into the CNS, brain, and/or spinal cord along an olfactory neural pathway. Typically such an embodiment includes administering the MC4-R agonist to tissue innervated by the olfactory nerve and inside the nasal cavity. The olfactory neural pathway innervates primarily the olfactory epithelium in the upper third of the nasal cavity, as described above. Olfactory neurons innervate this tissue and can provide a direct connection to the CNS, brain, and/or spinal cord due, it is believed, to their role in olfaction.

Delivery through the olfactory neural pathway can employ lymphatics that travel with the olfactory nerve to the olfactory bulb and other brain areas and from there into dural lymphatics associated with portions of the CNS. Therefore, transport along the olfactory nerve can also deliver an MC4-R agonist to an olfactory bulb. A perivascular pathway and/or a hemangiolymphatic pathway, such as lymphatic channels running within the adventitia of cerebral blood vessels, can provide an additional mechanism for transport of therapeutic MC4-R agonist(s) to the brain from tissue innervated by the olfactory nerve.

An MC4-R agonist can be administered to the olfactory nerve, for example, through the olfactory epithelium. Such administration can employ extracellular or intracellular (e.g., transneuronal) anterograde and retrograde transport of the agonist entering through the olfactory nerves to the brain and its meninges. Once the MC4-R agonist is dispensed into or onto tissue innervated by the olfactory nerve, the agonist may transport through the tissue and travel along olfactory neurons into areas of the CNS including the olfactory bulb, and cortical and subcortical structures.

Delivery through the olfactory neural pathway can employ movement of a MC4-R agonist into or across mucosa or epithelium into the olfactory nerve or into a lymphatic, a blood vessel perivascular space, a blood vessel adventitia, or a blood vessel lymphatic that travels with the olfactory nerve to the olfactory bulb and from there into meningial lymphatics associated with portions of the CNS, such as the frontal cortex and anterior olfactory nucleus. Blood vessel lymphatics include lymphatic channels that are around the blood vessels on the outside of the blood vessels. This also is referred to as the hemangiolymphatic system. Introduction of an MC4-R agonist into the blood vessel lymphatics does not necessarily introduce the agonist into the blood.

The Trigeminal Neural Pathway

One embodiment of the present method includes delivery of an MC4-R agonist to the subject in a manner such that the agonist is transported into the CNS, brain, and/or spinal cord along a trigeminal neural pathway. Typically, such an embodiment includes administration of the MC4-R agonist to a portion of the nasal cavity innervated by the trigeminal nerve, as described above. Trigeminal neurons innervate the nasal cavity and can provide a direct connection to the CNS, brain, and/or spinal cord due, it is believed, to their role in the common chemical sense including mechanical sensation, thermal sensation and nociception (for example detection of hot spices and of noxious chemicals).

Delivery through the trigeminal neural pathway can employ lymphatics that travel with the trigeminal nerve to the pons and other brain areas and from there into dural lymphatics associated with portions of the CNS, such as the spinal cord. Transport along the trigeminal nerve can also deliver MC4-R agonists to an olfactory bulb. A perivascular pathway and/or a hemangiolymphatic pathway, such as lymphatic channels running within the adventitia of cerebral blood vessels, can provide an additional mechanism for transport of therapeutic MC4-R agonists to the spinal cord from tissue innervated by the trigeminal nerve.

The trigeminal nerve includes large diameter axons, which mediate mechanical sensation (e.g., touch), and small diameter axons, which mediate pain and thermal sensation. The trigeminal nerve cell bodies are located in the semilunar (or trigeminal) ganglion or the mesencephalic trigeminal nucleus in the midbrain. Certain portions of the trigeminal nerve extend into the nasal cavity. Individual fibers of the trigeminal nerve collect into a large bundle, travel underneath the brain and enter the ventral aspect of the pons. An MC4-R agonist can be administered to the trigeminal nerve, for example through the mucosa and/or epithelium of the nasal cavity. Such administration can employ either cellular or intracellular (e.g., transneuronal) anterograde and retrograde transport of the MC4-R agonist entering through the trigeminal nerves to the brain and its meninges, to the brain stem, or to the spinal cord. Once the MC4-R agonist is dispensed into or onto tissue innervated by the trigeminal nerve, the agonist may transport through the tissue and travel along trigeminal neurons into areas of the CNS including the brain stem, cerebellum, spinal cord, olfactory bulb, and cortical and subcortical structures.

Delivery through the trigeminal neural pathway can employ movement of a MC4-R agonist across nasal mucosa or epithelium into the trigeminal nerve or into a lymphatic, a blood vessel perivascular space, a blood vessel adventitia, or a blood vessel lymphatic that travels with the trigeminal nerve to the pons and from there into meningial lymphatics associated with portions of the CNS such as thc spinal cord. Blood vessel lymphatics include lymphatic channels that are around the blood vessels on the outside of the blood vessels. This also is referred to as the hemangiolymphatic system. Introduction of an MC4-R agonist into the blood vessel lymphatics does not necessarily introduce the agonist into the blood.

Neural Pathways and Nasal Administration

In one embodiment, the method of the invention can employ delivery by a neural pathway (e.g., a trigeminal or olfactory neural pathway), after administration to the nasal cavity. Upon administration to the nasal cavity, delivery via the trigeminal neural pathway may employ movement of an MC4-R agonist through the nasal mucosa and/or epithelium to reach a trigeminal nerve, or a perivascular and/or lymphatic channel that travels with the nerve. Upon administration to the nasal cavity, delivery via the olfactory neural pathway may employ movement of an MC4-R agonist through the nasal mucosa and/or epithelium to reach the olfactory nerve or a perivascular and/or lymphatic channel that travels with the nerve. For example, the MC4-R agonist can be administered to the nasal cavity in a manner that employs extracellular or intracellular (e.g., transneuronal) anterograde and retrograde transport into and along the trigeminal and/or olfactory nerves to reach the brain, the brain stem, or the spinal cord. Once the MC4-R agonist is dispensed into or onto the nasal mucosa and/or epithelium innervated by the trigeminal and or olfactory nerve, the agonist may transport through the nasal mucosa and/or epithelium and travel along trigeminal and/or olfactory neurons into areas of the CNS including the brain stem, cerebellum, spinal cord, olfactory bulb, and cortical and subcortical structures. Alternatively, administration to the nasal cavity can result in delivery of an MC4-R agonist into a blood vessel perivascular space or a lymphatic that travels with the trigeminal and/or olfactory nerve to the pons, olfactory bulb, and other brain areas, and from there into meningeal lymphatics associated with portions of the CNS such as the spinal cord. Transport along the trigeminal and/or olfactory nerve may also deliver the agonist administered to the nasal cavity, to the olfactory bulb, midbrain, diencephalon, medulla and cerebellum. An agonist administered to the nasal cavity can enter the ventral dura of the brain and travel in lymphatic channels within the dura.

In addition, the method of the invention can be carried out in a way that employs a perivascular pathway and/or an hemangiolymphatic pathway, such as a lymphatic channel running within the adventitia of a cerebral blood vessel, to provide an additional mechanism for transport of the MC4-R agonist to the spinal cord from the nasal mucosa and/or epithelium. An MC4-R agonist transported by the hemangiolymphatic pathway does not necessarily enter the circulation. Blood vessel lymphatics associated with the circle of Willis, as well as blood vessels following the trigeminal and/or olfactory nerve can also be involved in the transport of the MC4-R agonist.

Administration to the nasal cavity employing a neural pathway can deliver a MC4-R agonist to the brain stem, cerebellum, spinal cord and cortical and subcortical structures. The MC4-R agonist alone may facilitate this movement into the CNS, brain, and/or spinal cord. Alternatively, the carrier or other transfer-promoting factors may assist in the transport of the MC4-R agonist into and along the trigeminal and/or olfactory neural pathway. Administration to the nasal cavity of a therapeutic MC4-R agonist can bypass the blood brain barrier through a transport system from the nasal mucosa and/or epithelium to the brain and spinal cord.

The method of the present invention includes administering an MC4-R agonist to the nasal cavity of a human or other mammal suffering from an MC4-R-mediated disorder. Some embodiments of the method of the present invention contemplate any biological disorder or disease in which MC4-R is implicated. Examples of such diseases include, but are not limited to, obesity, eating disorders, endocrine disorders such as type 11 diabetes, erectile disorders, cardiovascular disorders, neuronal injuries or disorders, inflammation, fever, cognitive disorders, and sexual behavior disorders. In a more specific embodiment, the instant invention provides compounds, compositions, and methods effective for reducing food and energy intake and body weight; reducing serum insulin and glucose levels; alleviating insulin resistance; and reducing serum levels of free fatty acids. Accordingly, the MC4-R agonists of the preferred embodiments of the instant invention are particularly effective in treating those disorders or diseases associated with obesity, type 11 diabetes, or eating disorders such as bulimia.

“Treating” within the context of the preferred embodiments of the instant invention, means any alleviation of any symptom associated with a disorder or disease, or any reduction of progression or worsening of symptoms, or prevention or prophylaxis of the disease or disorder. For example, within the context of obesity, successful treatment may include an alleviation of symptoms or halting the progression of the disease, as measured by reduction in body weight, or a reduction in amount of food or energy intake. Likewise, successful treatment of type I or type II diabetes may include an alleviation of symptoms or halting the progression of the disease, as measured by a decrease in serum glucose or insulin levels in, for example, hyperinsulinemic or hyperglycemic patients.

In one embodiment, the invention provides methods including delivering an MC4-R agonist (which may be a single agonist or combination of agonists) to pertinent areas of the brain and spinal cord through transport along neural pathways connecting the nasal cavity with the central nervous system. These neural pathways include the olfactory and trigeminal neural pathways described above. Transport along these pathways can occur not only along the nerves themselves, but also through perivascular and lymphatic channels that travel with the nerves. Delivery of the MC4-R agonist to the central nervous system by that transport system may be achieved in several ways, which are known to those having skill in the art of formulating and delivering substances by intranasal routes. One technique comprises delivering the MC4-R agonist alone to the nasal cavity. In this instance, the chemical characteristics of the agonist itself facilitate its transport to the appropriate neurons in the central nervous system. Alternatively, the MC4-R agonist may be combined with one or more other substances that assist transportation of the agonist to the pertinent sites in the brain. It is preferred that auxiliary substances are capable of delivering the MC4-R agonist to peripheral sensory neurons and/or along neural pathways to dysfunctioning areas of the brain and/or spinal cord.

More specific embodiments include those methods in which the MC4-R agonist (again, alone or as a combination of individual MC4-R agonists) is delivered to the upper third of the nasal cavity and particularly to the olfactory epithelium. Without wishing to be bound to any particular theory of action, such delivery is thought to promote transport of the agonist along the peripheral olfactory neurons into the central nervous system. Such embodiments of the invention provide transport of the MC4-R agonist to the brain and spinal cord by means of the nervous system even if the MC4-R agonist in question is unable to cross the blood-brain barrier.

Delivery of the MC4-R agonist along the olfactory and trigeminal neural pathways offers a number of advantages for treating MC4-R-mediated disorders. A significant advantage is that the olfactory and trigeminal systems provide a direct connection between the outside environment and the brain, thus providing quick and ready delivery of an MC4-R agonist for treatment of MC4-R disorders and MC4-R mediated diseases. Further, delivery along these neural pathways allows the therapeutic MC4-R agonist to reach the hypothalamus. In addition, because there is no need to achieve high levels of the drug in the circulation, systemic side effects can be reduced. Thus intranasal delivery along the neural pathways targets the central nervous system.

III. Pharmaceutical Composition

The MC4-R agonist administered by the method of the preferred embodiments of the invention may be generally absorbed into the bloodstream and/or the neural pathway(s) of the mammal. In one embodiment, a large enough quantity of a MC4-R agonist is applied at non-toxic levels sufficient to provide an effective level of activity within the neural system against the MC4-R disorder or MC4-R mediated disease. Such quantities can be determined using methods known to those having skill in the pharmaceutical and medical arts. The MC4-R agonist may be administered to the nasal cavity alone or in combination with one or more other agents that are effective in modulating neurologic and/or metabolic function(s). A single MC4-R agonist or a mixture of two or more MC4-R agonists may be administered in accordance with the present invention.

The method may employ a pharmaceutical composition capable of transporting the MC4-R agonist to the appropriate regions of the brain. Techniques for formulation and administration of drugs in general, may be found in the latest edition of “Remington's Pharmacological Sciences,” Mack Publishing Co., Easton, Pa. The pharmaceutical composition may comprise a pharmaceutically acceptable carrier. The carrier of the composition may be any material, which is otherwise compatible with the active ingredients of the composition. Where the carrier is a liquid, it is preferred that the carrier is hypotonic or isotonic with nasal fluids and within the range of pH 4.5-7.5. Where the carrier is in powdered form, it is preferred that the carrier is also within an acceptable non-toxic pH range.

The composition may be dispensed intranasally as a powdered or liquid nasal spray, suspension, nose drops, a gel or ointment, through a tube or catheter, by syringe, by packtail, by pledget, or by submucosal infusion. The compounds of the preferred embodiments of the present invention may be conveniently delivered in the form of an aerosol spray using a pressurized pack or a nebulizer and a suitable propellant, e.g., without limitation, dichlorodifluoromethane, trichlorofluoromethane, dichlorotetrafluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the compound and a suitable powder base such as lactose or starch. Examples of intranasal formulations and methods of administration can be found in PCT publications WO01/41782, WO00/33813, WO91/97947, and U.S. Pat. Nos. 6,180,603; 6,313,093; and 5,624,898. The latter-cited U.S. patents are incorporated herein by reference and for all purposes. A propellant for an aerosol formulation may include compressed air, nitrogen, carbon dioxide, or a hydrocarbon based low boiling solvent. The compound or compounds of the instant invention are conveniently delivered in the form of an aerosol spray presentation from a nebulizer or the like.

In a preferred embodiment, the MC4-R agonist is capable of at least partially dissolving in the fluids that are secreted by the mucous membrane that surround the cilia of the olfactory receptor cells of the olfactory epithelium in order to be absorbed into the olfactory neurons. Alternatively, the invention may combine the MC4-R agonist with a carrier and/or other substances that foster dissolution of the agonist within nasal secretions. Potential adjuvants include Captisol, GM-1, phosphatidylserine (PS), and emulsifiers such as polysorbate 80.

To further facilitate the transport of the MC4-R agonist along the olfactory neural pathway, the method of the preferred embodiments of the present invention may combine the agonist with substances that enhance the absorption of the agonist through the olfactory epithelium. It is preferred that the additives promote the transport of the agonist along the peripheral olfactory receptor neurons, which provide a direct connection between the brain and the outside environment due to their role in odor detection. IV. Dosage

The optimal concentration of the MC4-R agonist will necessarily depend upon the specific agonist used, the characteristics of the patient, and the nature the MC4-R disorder or the MC4-R-mediated disorder for which the treatment is sought. These factors can be determined by those of skill in the medical and pharmaceutical arts in view of the present disclosure. Generally, a therapeutically effective dose is desired. A therapeutically effective dose refers to that amount of the compound that results in a degree of amelioration of symptoms relative to the status of such symptoms prior to treatment. Specific dosages may be adjusted depending on conditions of disease, the age, brain size, body weight, general health conditions, sex, diet of the subject, dose intervals, administration routes, excretion rate, and combinations of drugs. Any of the above dosage forms containing effective amounts are well within the bounds of routine experimentation and therefore, well within the scope of the instant invention. A therapeutically effective dose may vary depending upon the route of administration and dosage form. The preferred compound or compounds of the instant invention is a formulation that exhibits a high therapeutic index. The therapeutic index is the dose ratio between toxic and therapeutic effects, which can be expressed as the ratio between LD₅₀ and ED₅₀. The LD₅₀ is the dose lethal to 50% of the population and the ED₅₀ is the dose therapeutically effective in 50% of the population. The LD₅₀ and ED₅₀ are determined by standard pharmaceutical procedures in animal cell cultures or experimental animals.

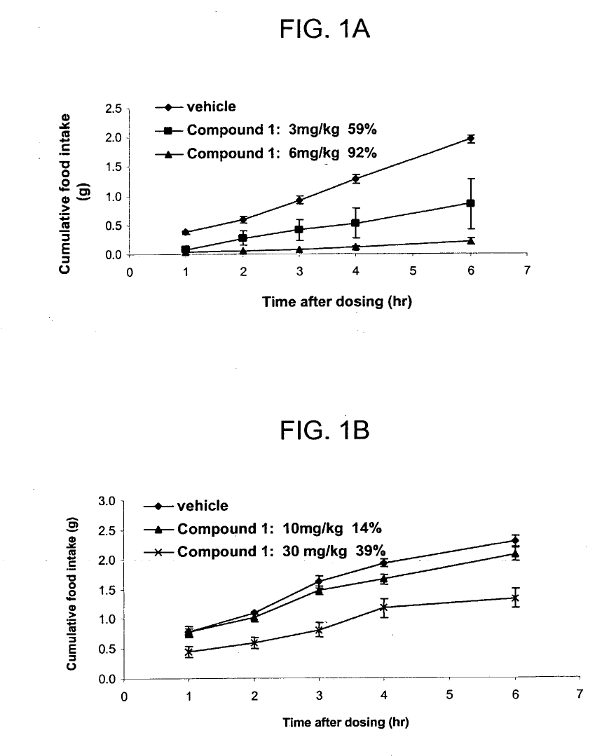

Surprisingly it has been found that the efficacy of the present compounds has been increased by intranasal administration. As can be seen in the figures, intranasal administration significantly increased the biological effect (decreased cumulative food intake) compared to oral administration of the same compound even though the oral dosage was ten times greater than the intranasal dosages. For example, in FIGS. 1A and 1B a dosage of 30 mg/kg given orally produced a 39% decrease in cumulative food intake whereas a 3 mg/kg dose of the same compound administered intranasally provided a 59% reduction in the same biological response thus giving an approximately 15 times greater biological effect as measured by the equation [(percent decrease in cumulative food intake for intranasal administration/percent decrease in cumulative food intake for oral administration) divided by (intranasal dosage/oral dosage)], i.e. ((59%/39%)÷(3/30))=approximately 15.1 times increase in biological effect. These results unexpectedly suggest that the efficacy of the present compounds is increased significantly, such as at least about 1.5, 2.5, 4, 5, 6, 7.5, 9, 10, 12 or 15 times, upon intranasal administration as compared to oral administration. Accordingly, the present methods can provide for intranasal administration of the present compounds in dosages that are on the order of at least about 1.5, 2.5, 4, 5, 6, 7.5, 9, 10, 12 or 15 times less than oral dosages while achieving the same or greater biological effect on the subject to which the compound is administered. In a similar manner, the present invention also provides compositions for intranasal administration that contain the present compounds in dosages that are less than at least about 1.5, 2.5, 4, 5, 6, 7.5, 9, 10, 12 or 15 times than those found in comparable oral dosage forms. In a further embodiment, the present invention can provide a device for intranasal administration, such as a nasal spray or inhaler, that contains a relatively large amount of the present compounds but that delivers individual dosages that are less than at least about 1.5, 2.5, 4, 5, 6, 7.5, 9, 10, 12 or 15 times than those found in comparable oral dosage forms.

V. Testing of MC4-R Agonists in vivo

Subjects: ob/ob mice; approximately 10 weeks old males; body weight 50-60 grams.

The mice were divided into the following groups:

1) Mice received only vehicle. The vehicle was one of the following: water, 10 mM phosphate buffer, 5% captisol in 10 mM phosphate buffer (see Table 1 for details).

2) Mice received a compound of an embodiment of the present invention at a dosage of 1 mg/kg.

3) Mice received a compound of an embodiment of the present invention at a dosage of 3 mg/kg.

4) Mice received a compound of an embodiment of the present invention at a dosage of 6 mg/kg. n=6-8 animals per group

Note: For compound 1 and compound 2, the 1 mg/kg group was not included.

TABLE 1

| Compound # | Vehicle |
|---|---|
| 1 | Water |
| 2 | 10 mM phosphate |
| 3 | Water |
| 4 | 10 mM phosphate |
| 5 | 10 mM phosphate |
| 6 | 5% captisol in 10 mM phosphate |
| 7 | Water |
| 8 | Water |
| 9 | Water |
| 10 | Water |
| 11 | Water |
| 12 | Water |
| 13 | Water |

Generals Procedure:

Animals were fasted overnight (˜16 hours), and an MC4-R agonist compound as described in Tables 1-3 was administered at 8:30 am next day. The administration volume was 20-25 pl/50 grams of body weight. The animal was gently held by one hand, and the compound solution was delivered by the other hand using a pipette with fine tips. The rate of delivery was such that the full amount was given in not less than 60 seconds to ensure maximal absorption. Pre-weighed food was given to the animals immediately after dosing, and animals had free access to water throughout the study. Food weighing was performed at 1, 2, 3, 4, and 6 hours after dosing. On occasion, food weighing was also taken at the 8 hour or 24 hour time point. Animals were euthanized using carbon dioxide followed by cervical dislocation at the end of the experiment.

Results:

TABLE 2

| Compound | | |
|---|---|---|
| Number | Dosage (mg/kg) | % Food Intake Reduction at 4 hour |
| 1 | 3 | 59.0 |
| 1 | 6 | 91.5 |
| 2 | 3 | 23.7 |
| 2 | 6 | 1.7 |
| 3 | 1 | −3.0 |
| 3 | 3 | 63.8 |
| 3 | 6 | 70.3 |
| 4 | 1 | 12.0 |
| 4 | 3 | 77.9 |
| 4 | 6 | 70.2 |
| 5 | 1 | 10.4 |
| 5 | 3 | 53.1 |
| 5 | 6 | 67.3 |
| 6 | 1 | −11.9 |
| 6 | 3 | 21.3 |
| 6 | 6 | 57.4 |
| 7 | 1 | 23.5 |
| 7 | 3 | 42.7 |
| 7 | 6 | 85.5 |
| 8 | 1 | 37.8 |
| 8 | 3 | 60.0 |
| 8 | 6 | 84.1 |
| 9 | 1 | 35.2 |
| 9 | 3 | 70.2 |
| 9 | 6 | 69.6 |
| 10 | 1 | 14.2 |
| 10 | 3 | 39.7 |
| 11 | 0.5 | −3.8 |
| 11 | 1 | 27.7 |
| 11 | 3 | 82 |
| 12 | 0.5 | 6 |
| 12 | 1 | 22.6 |
| 12 | 3 | 47.2 |

TABLE 3

| Compound | |
|---|---|
| Number | Molecular Structure |
| 1 | 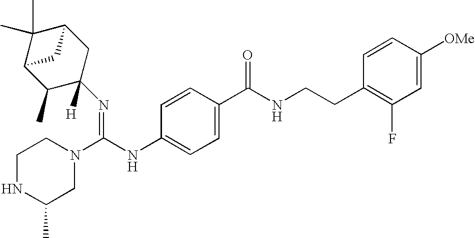 |
| 2 | 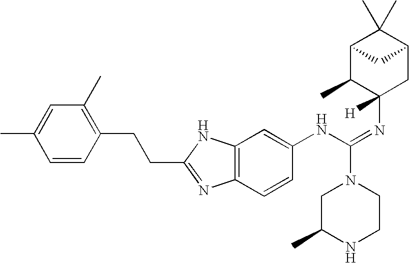 |
| 3 | 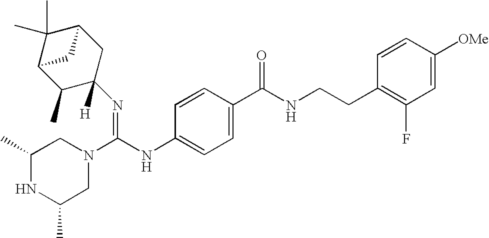 |
| 4 | 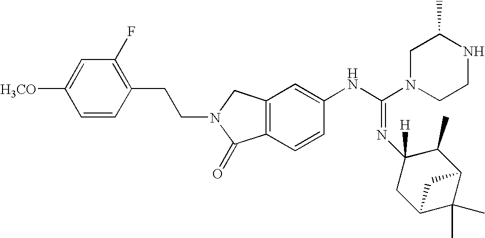 |
| 5 | 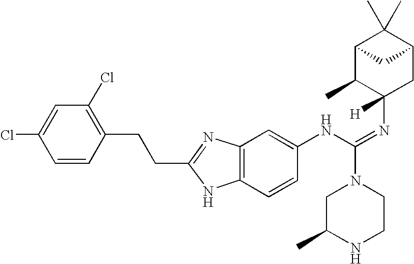 |
| 6 | 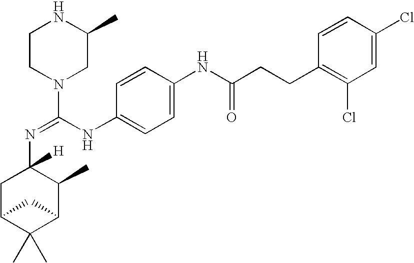 |
| 7 | 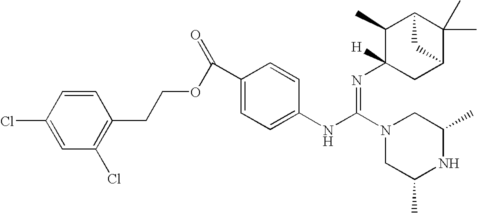 |
| 8 | 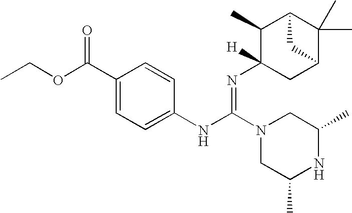 |
| 9 | 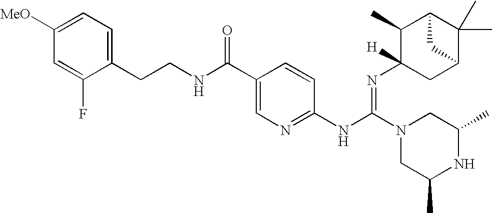 |
| 10 | 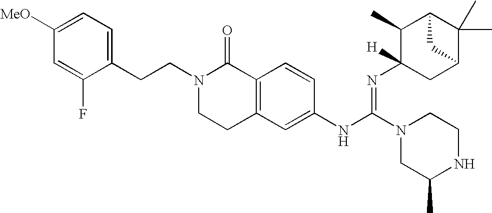 |
| 11 | 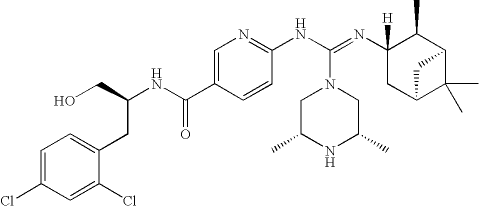 |
| 12 | 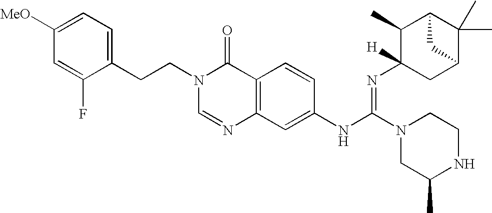 |
| 13 | 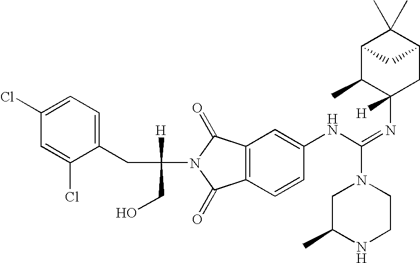 |

### EXAMPLE 1A

### Intransal (IN) Efficacy of Compound 1 Ob/ob Mice, Together with Simplified PKs

Subjects:

ob/ob mice, ˜10 weeks old males. Body weight 50-60 grams.

IN efficancy groups:

| | |
|---|---|
| 1) | Vehicle = water |
| 2) | Compound 1 = 3 mg/kg |
| 3) | Compound 1 = 6 mg/kg |
| | n = 6/group fasted |
| 4) | PK group of Compound 1 = 6 mg/kg |
| | n = 3 fasted |

IN Efficacy Procedure:

Mice were fasted overnight. At 8:30 am the next morning, they were dosed with 25 μl of vehicle or compound solution by intranasal delivery. The solution was delivered using a pipette with protein loading tips. The rate of delivery was such that the full amount was given in not less than 60 seconds. Mice were fed with pre-weighed food immediately after dosing, and had access to water the entire time. Food weight was measured at 1, 2, 3, 4, 6, and 8 hours following the dosing. Mice were euthanized at the end of the study using CO₂ followed by cervical dislocation.

PK Procedure: Animals were dosed the same as the efficacy groups, and food was given right after dosing. Tails were anesthetized with topical EMLA cream approximately 15 to 30 minutes prior to initial tail snip. Approximately 30 μl blood samples were collected by tail snipping at 5, 10, 60, and 180 minutes following dosing. Plasma samples, together with ˜2 mg dry powder of the compound were analyzed upon finishing of the study. Alternatively, samples were kept at -20 C until they could be run together with other PK samples. Animals were euthanized using CO₂ followed by cervical dislocation after the last blood sample had been collected.

### EXAMPLE 1B

### Oral (PO) Efficacy of Compound 1 in Ob/ob Mice,

Subjects:

ob/ob mice, ˜10 weeks old males. Body weight ˜50 grams.

PO Efficacy Groups:

| | |
|---|---|
| 1) | Vehicle = water |
| 2) | Compound 1 = 200 μl of 2.5 mg/ml (10 mg/kg) |
| 3) | Compound 1 = 200 μl of 7.5 mg/ml (30 mg/kg) |
| | n = 8/group fasted |

PO Efficacy Procedure:

Mice were fasted overnight. At about 9:00 am the next morning, they were dosed with 200 μl of vehicle or compound solution by oral gavage. Mice were fed with pre-weighed food immediately after dosing, and had access to water the entire time. Food weight was measured at 1, 2, 3, 4, 6, and 24 hours following the dosing.

### EXAMPLE 2

### Intranasal (IN) Efficacy of Compound 2 in Ob/ob Mice, Together with Simplified PKs

Subjects:

ob/ob mice, ˜10 weeks old males. Body weight 50-60 grams.

IN Efficacy Groups:

| | |
|---|---|
| 1) | Vehicle = 10 mM phosphate |
| 2) | Compound 2 = 3 mg/kg |
| 3) | Compound 2 = 6 mg/kg |
| | n = 6/group fasted |
| 4) | PK group of Compound 2 = 6 mg/kg |
| | n = 3 fasted |

IN Efficacy Procedure:

Mice were fasted overnight. At 8:30 am the next morning, they were dosed with 25 μl of vehicle or compound solution by intranasal delivery. The solution was delivered using a pipette with protein loading tips. The rate of delivery was such that the full amount was given in not less than 60 seconds. Mice were fed with pre-weighed food immediately after dosing, and had access to water the entire time. Food weight was measured at 1, 2, 3, 4, 6, and 8 hours following the dosing. Mice were euthanized at the end of the study using CO₂ followed by cervical dislocation.

PK Procedure:

Animals were dosed the same as the efficacy groups, and food was given right after dosing. Tails were anesthetized with topical EMLA cream approximately 15 to 30 minutes prior to initial tail snip. Approximately 30 μl blood samples were collected by tail snipping at 5, 10, 60, and 180 minutes following dosing. Plasma samples, together with ˜2 mg dry powder of the compound were analyzed upon finishing of the study. Alternatively, samples were kept at −20 ° C. until they could be run together with other PK samples. Animals were euthanized using CO2 followed by cervical dislocation after the last blood sample had been collected.

### EXAMPLE 3

### Intranasal (IN) Efficacy of Compound 3 in Ob/ob Mice, Together with Simplified PKs

Subjects:

ob/ob mice, 10 weeks old males. Body weight 50 grams.

IN Efficacy Groups:

| | |
|---|---|
| 1) | Vehicle = water |
| 2) | Compound 3 = 25 μl of 2 mg/ml (1 mg/kg) |
| 3) | Compound 3 = 25 μl of 6 mg/ml (3 mg/kg) |
| 4) | Compound 3 = 25 μl of 12 mg/ml (6 mg/kg) |
| | n = 6/group fasted |
| 5) | PK group of Compound 3 = 25 μl of 12 mg/ml (6 mg/kg) |
| | n = 3 fasted |

IN Efficacy Procedure:

Mice were fasted overnight. At 8:30 am the next morning, they were dosed with 25 μl of vehicle or compound solution by intranasal delivery. The solution was delivered using a pipette with protein loading tips. The rate of delivery was such that the full amount was given in not less than 60 seconds. Mice were fed with pre-weighed food immediately after dosing, and had access to water the entire time. Food weight was measured at 1, 2, 3, 4, 6, and 8 hours following the dosing. Mice were euthanized at the end of the study using CO₂ followed by cervical dislocation.

PK Procedure:

Animals were dosed the same as the efficacy groups, and food was given right after dosing. Tails were anesthetized with topical EMLA cream approximately 15 to 30 minutes prior to initial tail snip. Approximately 30 μl blood samples were collected by tail snipping at 5, 10, 60, and 180 minutes following dosing. Plasma samples, together with ˜2 mg dry powder of the compound were analyzed upon finishing of the study. Alternatively, samples were kept at −20 ° C. until they could be run together with other PK samples. Animals were euthanized using CO₂ followed by cervical dislocation after the last blood sample had been collected.

### EXAMPLE 4

### Intranasal (IN) Efficacy of Compound 4 in Ob/ob Mice, Together with Simplified PKs

Subjects:

ob/ob mice, ˜10 weeks old males. Body weight ˜50 grams.

IN Efficacy Groups:

| | |
|---|---|
| 1) | Vehicle = 10 mM phosphate |
| 2) | Compound 4 = 25 μl of 2 mg/ml (1 mg/kg) |
| 3) | Compound 4 = 25 μl of 6 mg/ml (3 mg/kg) |
| 4) | Compound 4 = 25 μl of 12 mg/ml (6 mg/kg) |
| | n = 8/group fasted |
| 5) | PK group of Compound 4 = 25 μl of 12 mg/ml (6 mg/kg) |
| | n = 3 fasted |

IN Efficacy Procedure: Mice were fasted overnight. At 8:30 am the next morning, they were dosed with 25 μl of vehicle or compound solution by intranasal delivery. The solution was delivered using a pipette with protein loading tips. The rate of delivery was such that the full amount was given in not less than 60 seconds. Mice were fed with pre-weighed food immediately after dosing, and had access to water the entire time. Food weight was measured at 1, 2, 3, 4, 6, and 8 hours following the dosing.

PK Procedure:

Animals were dosed the same as the efficacy groups, and food was given right after dosing. Tails were anesthetized with topical EMLA cream approximately 15 to 30 minutes prior to initial tail snip. Approximately 50 μl blood samples were collected by tail snipping at 15, 60, and 180 minutes following dosing. Plasma samples, together with ˜2 mg dry powder of the compound were analyzed upon finishing of the study. All animals were euthanized by CO₂ followed by cervical dislocation at the end of the study.

### EXAMPLE 5

### Intranasal (IN) Efficacy of Compound 5 in Ob/ob Mice, Together with Simplified PKs

Subjects:

ob/ob mice, 10 weeks old males. Body weight 50 grams.

IN Efficacy Groups:

| | |
|---|---|
| 1) | Vehicle = 10 mM phosphate |
| 2) | Compound 5 = 25 μl of 2 mg/ml (1 mg/kg) |
| 3) | Compound 5 = 25 μl of 6 mg/ml (3 mg/kg) |
| 4) | Compound 5 = 25 μl of 12 mg/ml (6 mg/kg) |
| | n = 8/group fasted |
| 5) | PK group of Compound 5 = 25 μl of 12 mg/ml (6 mg/kg) |
| | n = 3 fasted |

IN Efficacy Procedure:

Mice were fasted overnight. At 8:30 am the next morning, they were dosed with 25 μl of vehicle or compound solution by intranasal delivery. The solution was delivered using a pipette with protein loading tips. The rate of delivery was such that the full amount was given in not less than 60 seconds. Mice were fed with pre-weighed food immediately after dosing, and had access to water the entire time. Food weight was measured at 1, 2, 3, 4, 6, and 8 hours following the dosing.

PK Procedure:

Animals were dosed the same as the efficacy groups, and food was given right after dosing. Tails were anesthetized with topical EMLA cream approximately 15 to 30 minutes prior to initial tail snip. Approximately 50 μl blood samples were collected by tail snipping at 15, 60, and 180 minutes following dosing. Plasma samples, together with ˜2 mg dry powder of the compound were analyzed upon finishing of the study. All animals were euthanized by CO₂ followed by cervical dislocation at the end of the study.

### EXAMPLE 6

### Intranasal (IN) Efficacy of Compound 6 in Ob/ob Mice, Together with Simplified PKs

Subjects:

ob/ob mice, ˜10 weeks old males. Body weight ˜50 grams.

IN Efficacy Groups:

| | |
|---|---|
| 1) | Vehicle = 5% captisol and 10 mM phosphate |
| 2) | Compound 6 = 20 μl of 2.5 mg/ml (1 mg/kg) |
| 3) | Compound 6 = 20 μl of 7.5 mg/ml (3 mg/kg) |
| 4) | Compound 6 = 20 μl of 12.5 mg/ml (6 mg/kg) |
| | n = 8/group fasted |
| 5) | PK group of 143804 = 20 μl of 12 mg/ml (6 mg/kg) |
| | n = 3 fasted |

IN Efficacy Procedure:

Mice were fasted overnight. At 8:30 am the next morning, they were dosed with 20 μl of vehicle or compound solution by intranasal delivery. The solution was delivered using a pipette with protein loading tips. The rate of delivery was such that the full amount was given in not less than 60 seconds. Mice were fed with pre-weighed food immediately after dosing, and had access to water the entire time. Food weight was measured at 1, 2, 3, 4, 6, and 8 hours following the dosing.

PK Procedure:

Animals were dosed the same as the efficacy groups, and food was given right after dosing. Tails were anesthetized with topical EMLA cream approximately 15 to 30 minutes prior to initial tail snip. Approximately 50 μl blood samples were collected by tail snipping at 15, 60, and 180 minutes following dosing. Plasma samples, together with ˜2 mg dry powder of the compound were analyzed upon finishing of the study. All animals were euthanized by CO₂ followed by cervical dislocation at the end of the study.

### EXAMPLE 7

### Intranasal (IN) Efficacy of Compound 7 in Ob/ob Mice, Together with Simplified PKs

Subjects:

ob/ob mice, ˜10 weeks old males. Body weight ˜50 grams.

IN Efficacy Groups:

| | |
|---|---|
| 1) | Vehicle = water |
| 2) | Compound 7 = 20 μl of 2.5 mg/ml (1 mg/kg) |
| 3) | Compound 7 = 20 μl of 7.5 mg/ml (3 mg/kg) |
| 4) | Compound 7 = 20 μl of 12.5 mg/ml (6 mg/kg) |
| | n = 8/group fasted |
| 5) | PK group of Compound 7 = 20 μl of 12.5 mg/ml (6 mg/kg) |
| | n = 3 fasted |

IN Efficacy Procedure:

Mice were fasted overnight. At 8:30 am the next morning, they were dosed with 20 μl of vehicle or compound solution by intranasal delivery. The solution was delivered using a pipette with protein loading tips. The rate of delivery was such that the full amount was given in not less than 60 seconds. Mice were fed with pre-weighed food immediately after dosing, and had access to water the entire time. Food weight was measured at 1, 2, 3, 4, 6, and 8 hours following the dosing.

PK Procedure:

Animals were dosed the same as the efficacy groups, and food was given right after dosing. Tails were anesthetized with topical EMLA cream approximately 15 to 30 minutes prior to initial tail snip. Approximately 50 μl blood samples were collected by tail snipping at 15, 60, and 180 minutes following dosing. Plasma samples, together with ˜2 mg dry powder of the compound were analyzed upon finishing of the study. All animals were euthanized by CO₂ followed by cervical dislocation at the end of the study.

### EXAMPLE 8

### Intranasal (IN) Efficacy of Compound 8 in Ob/ob Mice, Together with Simplified PKs

Subjects:

ob/ob mice, 10 weeks old males. Body weight 50 grams.

IN Efficacy Groups:

| | |
|---|---|
| 1) | Vehicle = water |
| 2) | Compound 8 = 25 μl of 2 mg/ml (1 mg/kg) |
| 3) | Compound 8 = 25 μl of 6 mg/ml (3 mg/kg) |
| 4) | Compound 8 = 25 μl of 12 mg/ml (6 mg/kg) |
| | n = 8/group fasted |
| 5) | PK group of Compound 8 = 25 μl of 12 mg/ml (6 mg/kg) |
| | n = 3 fasted |

IN Efficacy Procedure:

Mice were fasted overnight. At 8:30 am the next morning, they were dosed with 25 μl of vehicle or compound solution by intranasal delivery. The solution was delivered using a pipette with protein loading tips. The rate of delivery was such that the full amount was given in not less than 60 seconds. Mice were fed with pre-weighed food immediately after dosing, and had access to water the entire time. Food weight was measured at 1, 2, 3, 4, 6, and 8 hours following the dosing.

PK Procedure:

Animals were dosed the same as the efficacy groups, and food was given right after dosing. Tails were anesthetized with topical EMLA cream approximately 15 to 30 minutes prior to initial tail snip. Approximately 50 μl blood samples were collected by tail snipping at 5,10, 60, and 180 minutes following dosing. Plasma samples, together with ˜2 mg dry powder of the compound were analyzed upon finishing of the study. All animals were euthanized by CO₂ followed by cervical dislocation at the end of the study.

### EXAMPLE 9A

### Intranasal (IN) Efficacy of Compound 9 in Ob/ob Mice,

Subjects:

ob/ob mice, ˜10 weeks old males. Body weight 50 grams.

IN Efficacy Groups:

| | |
|---|---|
| 1) | Vehicle = water |
| 2) | Compound 9 = 25 μl of 2 mg/ml (1 mg/kg) |
| 3) | Compound 9 = 25 μl of 6 mg/ml (3 mg/kg) |
| | n = 8/group fasted |

IN Efficacy Procedure:

Mice were fasted overnight. At 8:30 am the next morning, they were dosed with 25 μl of vehicle or compound solution by intranasal delivery. The solution was delivered using a pipette with protein loading tips. The rate of delivery was such that the full amount was given in not less than 60 seconds. Mice were fed with pre-weighed food immediately after dosing, and had access to water the entire time. Food weight was measured at 1, 2, 3, 4, and 6 hours following the dosing.

### EXAMPLE 9B

### Oral (PO) Efficacy of Compound 9 in Ob/ob Mice,

Subjects:

ob/ob mice, ˜10 weeks old males. Body weight ˜50 grams.

PO Efficacy Groups:

| | |
|---|---|
| 1) | Vehicle = water |
| 2) | Compound 9 = 200 μl of 2.5 mg/ml (10 mg/kg) |
| 3) | Compound 9 = 200 μl of 7.5 mg/ml (30 mg/kg) |
| | n = 8/group fasted |

PO Efficacy Procedure:

Mice were fasted overnight. At about 9:00 am the next morning, they were dosed with 200 μl of vehicle or compound solution by oral gavage. Mice were fed with pre-weighed food immediately after dosing, and had access to water the entire time. Food weight was measured at 1, 2, 3, 4, 6, and 24 hours following the dosing.

### EXAMPLE 10

### Intranasal (IN) Efficacy of Compound 10 in Ob/ob Mice, Together with Simplified PKs

Subjects:

ob/ob mice, ˜10 weeks old males. Body weight ˜50 grams.

IN Efficacy Groups:

| | |
|---|---|
| 1) | Vehicle = water |
| 2) | Compound 10 = 25 μl of 2 mg/ml (1 mg/kg) |
| 3) | Compound 10 = 25 μl of 6 mg/ml (3 mg/kg) |
| | n = 8/group fasted |
| 4) | PK group of Compound 10 = 25 μl of 6 mg/ml (3 mg/kg) |
| | n = 3 fasted |

IN Efficacy Procedure:

Mice were fasted overnight. At 8:30 am the next morning, they were dosed with 25 μl of vehicle or compound solution by intranasal delivery. The solution was delivered using a pipette with protein loading tips. The rate of delivery was such that the full amount was given in not less than 60 seconds. Mice were fed with pre-weighed food immediately after dosing, and had access to water the entire time. Food weight was measured at 1, 2, 3, 4, 6, and 8 hours following the dosing.

PK Procedure:

Animals were dosed the same as the efficacy groups, and food was given right after dosing. Tails were anesthetized with topical EMLA cream approximately 15 to 30 minutes prior to initial tail snip. Approximately 50 μl blood samples were collected by tail snipping at 5,10, 60, and 180 minutes following dosing. Plasma samples, together with ˜2 mg dry powder of the compound were analyzed upon finishing of the study. All animals were euthanized by CO₂followed by cervical dislocation at the end of the study.

### EXAMPLE 11

### Intranasal (IN) Efficacy of Compound 11 in Ob/ob Mice, Together with Simplified PKs

Subjects:

ob/ob mice, ˜10 weeks old males. Body weight ˜50 grams.

IN Efficacy Groups:

| | |
|---|---|
| 1) | Vehicle = water |
| 2) | Compound 11 = 25 μl of 1 mg/ml (0.5 mg/kg) |
| 3) | Compound 11 = 25 μl of 2 mg/ml (1 mg/kg) |
| 4) | Compound 11 = 25 μl of 6 mg/ml (3 mg/kg) |
| | n = 8/group fasted |
| 5) | PK group of Compound 11 = 25 μl of 6 mg/ml (3 mg/kg) |
| | n = 3 fasted |

IN Efficacy Procedure:

Mice were fasted overnight. At 8:30 am the next morning, they were dosed with 25 μl of vehicle or compound solution by intranasal delivery. The solution was delivered using a pipette with protein loading tips. The rate of delivery was such that the full amount was given in not less than 60 seconds. Mice were fed with pre-weighed food immediately after dosing, and had access to water the entire time. Food weight was measured at 1, 2, 3, 4, 6, and 8 hours following the dosing.

PK Procedure:

Animals were dosed the same as the efficacy groups, and food was given right after dosing. Tails were anesthetized with topical EMLA cream approximately 15 to 30 minutes prior to initial tail snip. Approximately 50 μl blood samples were collected by tail snipping at 5,10, 60, and 180 minutes following dosing. Plasma samples, together with ˜2 mg dry powder of the compound were analyzed upon finishing of the study. All animals were euthanized by CO₂ followed by cervical dislocation at the end of the study.

### EXAMPLE 12

### Intranasal (IN) Efficacy of Compound 12 in Ob/ob Mice, Together with Simplified PKs

Subjects:

ob/ob mice, ˜10 weeks old males. Body weight ˜50 grams.

IN Efficacy Groups:

| | |
|---|---|
| 1) | Vehicle = water |
| 2) | Compound 12 = 25 μl of 1 mg/ml (0.5 mg/kg) |
| 3) | Compound 12 = 25 μl of 2 mg/ml (1 mg/kg) |
| 4) | Compound 12 = 25 μl of 6 mg/ml (3 mg/kg) |
| | n = 8/group fasted |
| 5) | PK group of Compound 12 = 25 μl of 6 mg/ml (3 mg/kg) |
| | n = 3 fasted |

IN Efficacy Procedure:

Mice were fasted overnight. At 8:30 am the next morning, they were dosed with 25 μl of vehicle or compound solution by intranasal delivery. The solution was delivered using a pipette with protein loading tips. The rate of delivery was such that the full amount was given in not less than 60 seconds. Mice were fed with pre-weighed food immediately after dosing, and had access to water the entire time. Food weight was measured at 1, 2, 3, 4, 6, and 8 hours following the dosing.

PK Procedure:

Animals were dosed the same as the efficacy groups, and food was given right after dosing. Tails were anesthetized with topical EMLA cream approximately 15 to 30 minutes prior to initial tail snip. Approximately 50 μl blood samples were collected by tail snipping at 5,10, 60, and 180 minutes following dosing. Plasma samples, together with ˜2 mg dry powder of the compound were analyzed upon finishing of the study. All animals were euthanized by C₂ followed by cervical dislocation at the end of the study.

### EXAMPLE 13A

### Intranasal (IN) Efficacy of Compound 13 in Ob/ob Mice,

Subjects:

ob/ob mice, ˜10 weeks old males. Body weight ˜50 grams.

IN Efficacy Groups:

| | |
|---|---|
| 1) | Vehicle = water |
| 2) | Compound 13 = 25 μl of 2 mg/ml (1 mg/kg) |
| 3) | Compound 13 = 25 μl of 6 mg/ml (3 mg/kg) |
| | n = 8/group fasted |

IN Efficacy Procedure:

Mice were fasted overnight. At 8:30 am the next morning, they were dosed with 25 μl of vehicle or compound solution by intranasal delivery. The solution was delivered using a pipette with protein loading tips. The rate of delivery was such that the full amount was given in not less than 60 seconds. Mice were fed with pre-weighed food immediately after dosing, and had access to water the entire time. Food weight was measured at 1, 2, 3, 4, and 6 hours following the dosing.

### EXAMPLE 13B

### Oral (PO) Efficacy of Compound 13 in Ob/ob Mice,

Subjects:

ob/ob mice, ˜10 weeks old males. Body weight ˜50 grams.

PO Efficacy Groups:

| | |
|---|---|
| 1) | Vehicle water |
| 2) | Compound 13 = 200 μl of 2.5 mg/ml (10 mg/kg) |
| 3) | Compound 13 = 200 μl of 7.5 mg/ml (30 mg/kg) |
| | n = 8/group fasted |

PO Efficacy Procedure:

Mice were fasted overnight. At about 9:00 am the next morning, they were dosed with 200 μl of vehicle or compound solution by oral gavage. Mice were fed with pre-weighed food immediately after dosing, and had access to water the entire time. Food weight was measured at 1, 2, 3, 4, 6, and 24 hours following the dosing.

It is understood that the invention is not limited to the embodiments specifically set forth herein for illustration, but embraces all such forms thereof as would be understood by one of skill in the art and come within the scope of the following claims.

What is claimed is:

1. A method for delivering a melanocortin-4 receptor agonist to a mammalian subject, comprising: administering an amount of the melanocortin-4 receptor agonist to a tissue inside the nasal cavity or sinuses of the mammalian subject, wherein the amount of the melanocortin-4 receptor agonist admininstered to the tissue inside the nasal cavity or sinuses is at least 1.5 times less than an amount required to achieve an equivalent effect when administered orally.

2. The method of claim 1, wherein the melanocortin-4 receptor agonist comprises a guanidine group.

3. The method of claim 1, wherein the the melanocortin-4 receptor agonist has a molecular weight of less than 900 grams mole.

4. The method of claim 1, wherein the molecular weight of the compound ranges from 450 grams per mole to 700 grams per mole.

5. The method of claim 1, wherein the melanocortin-4 receptor agonist comprises 3 or less amino acid residues.

6. The method of claim 1, wherein the melanocortin-4 receptor is not a peptide.

7. The method of claim 1, wherein the mammalian subject is a human.

8. The method of claim 7, wherein the human has a melanocortin-4 receptor mediated disease selected from obesity, an eating disorder, or type 11 diabetes.

9. The method of claim 1, further comprising administering the melanocortin-4 receptor agonist to the upper third of the nasal cavity.

10. The method of claim 9, wherein the melanocortin-4 receptor agonist is administered to the olfactory epithelium. 1.1

11. The method of claim 1, wherein the melanocortin-4 receptor agonist is administered as a powder or liquid nasal spray, as a suspension, as nose drops, as a gel or ointment, through a tube or catheter, by syringe, by packtail, by pledget, or by submucosal infusion.

12. The method of claim 1, wherein the melanocortin-4 receptor agonist is administered using an aerosol spray.

13. The method of claim 1, wherein the melanocortin-4 receptor agonist is administered as part of a pharmaceutical formulation that comprises the melanocortin-4 receptor agonist and a carrier.

14. The method of claim 1, wherein the amount of the melanocortin-4 receptor agonist admininstered to the tissue inside the nasal cavity or sinuses is at least 2.5 times less than the amount required to achieve the equivalent effect when administered orally.

15. The method of claim 1, wherein the amount of the melanocortin-4 receptor agonist admininstered to the tissue inside the nasal cavity or sinuses is at least 4.0 times less than the amount required to achieve the equivalent effect when administered orally.

16. The method of claim 1, wherein the amount of the melanocortin-4 receptor agonist admininstered to the tissue inside the nasal cavity or sinuses is at least 5.0 times less than the amount required to achieve the equivalent effect when administered orally.

17. The method of claim 1, wherein the amount of the melanocortin-4 receptor agonist admininstered to the tissue inside the nasal cavity or sinuses is at least 10.0 times less than the amount required to achieve the equivalent effect when administered orally.

18. The method of claim 1, wherein the amount of the melanocortin-4 receptor agonist admininstered to the tissue inside the nasal cavity or sinuses is at least 12.0 times less than the amount required to achieve the equivalent effect when administered orally. 1.1

19. The method of claim 1, wherein the melanocortin-4 receptor agonist is selected from 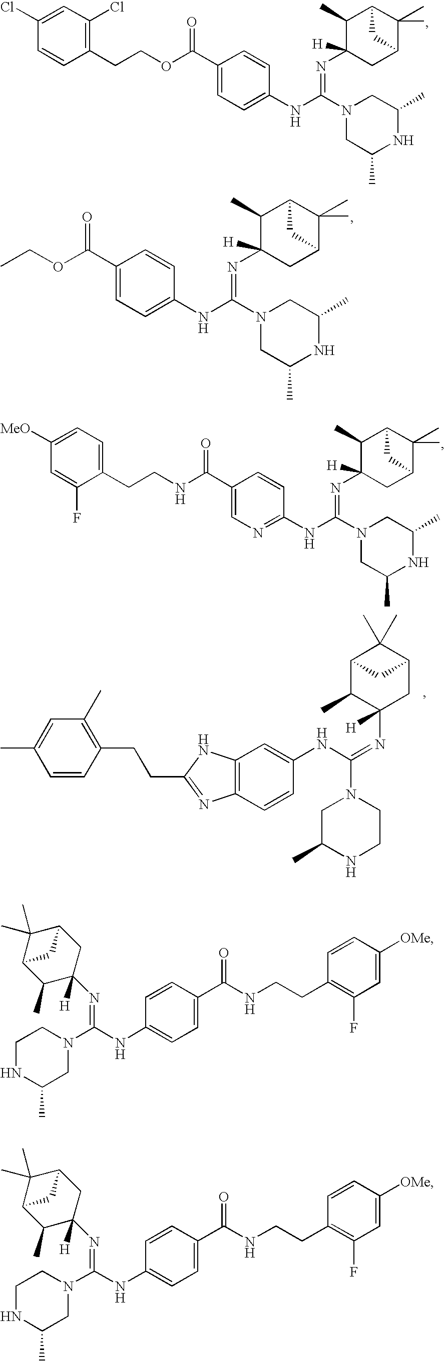 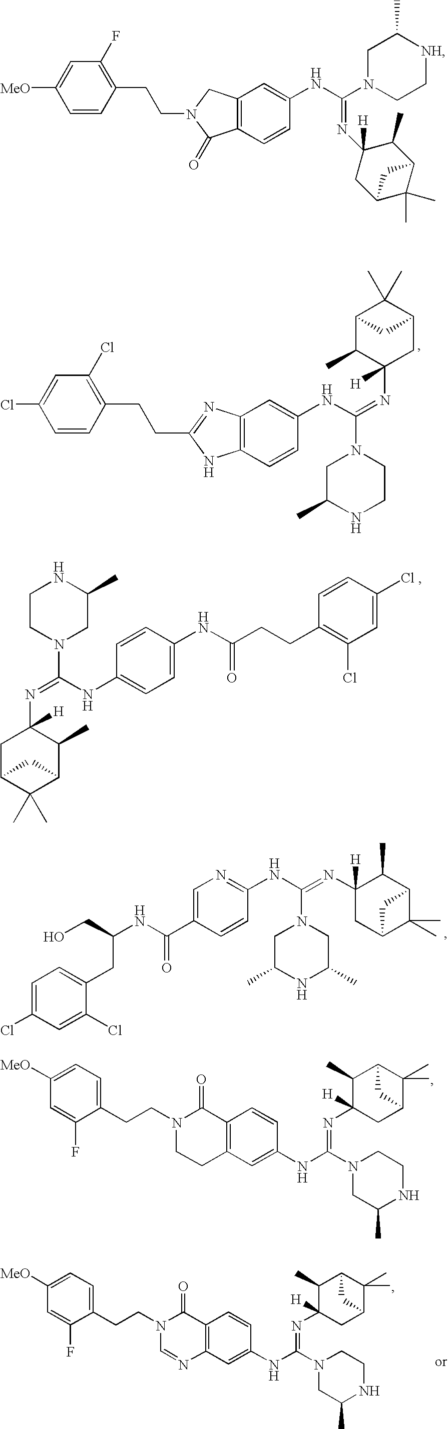 [chemical structure]
